(19) United States
(12) Patent Application Publication
(10) Pub. No.: US 2023/0255237 A1
(43) Pub. Date: Aug. 17, 2023

(54) METHODS AND COMPOSITIONS FOR IMPROVING FOOD SAFETY AND ENHANCING FEED EFFICIENCY IN POULTRY

(71) Applicant: BACTANA CORP., Farmington, CT (US)

(72) Inventors: John A. KALLASSY, West Hartford, CT (US); Simon A. MCMANUS, West Hartford, CT (US)

(21) Appl. No.: 18/175,244

(22) Filed: Feb. 27, 2023

Related U.S. Application Data
(63) Continuation-in-part of application No. 17/652,274, filed on Feb. 23, 2022.
(60) Provisional application No. 63/152,806, filed on Feb. 23, 2021.

(51) Int. Cl. A23K 10/18; A23K 50/75; A61P 31/04; A61K 35/741; A23K 30/20; A61K 9/00

(52) U.S. Cl. CPC A23K 10/18; A23K 30/20; A23K 50/75; A61K 9/0056; A61K 35/741; A61P 31/04

(57) ABSTRACT

The disclosure provides methods for improving feed efficiency in poultry, reducing morbidity and/or mortality in poultry from gastrointestinal disease, and reducing pathogen load in poultry and/or reducing pathogen contamination of meat or eggs from poultry, comprising administering Faecalibacterium prausnitzii or compositions made therefrom, e.g., an extract from a culture of Faecalibacterium prausnitzii, or a composition comprising optionally dried live cell or cell components and/or supernatant from a Faecalibacterium prausnitzii culture (FPS) to the poultry; together with compositions useful in said methods, and methods of making the same.

### CROSS-REFERENCE TO RELATED APPLICATIONS

This application is a continuation-in-part application, under 35 U.S.C. § 111, of U.S. Pat. Application No. 17/652,274, filed on Feb. 23, 2022, which claims priority to, and the benefit of, U.S. Provisional Application No. 63/152,806, filed on Feb. 23, 2021, the contents of each of which are hereby incorporated by reference in their entireties.

### FIELD

The disclosure relates to methods and compositions for improving growth, feed efficiency, and food safety in domestic poultry livestock production, using Faecalibacterium prausnitzii or compositions made therefrom, e.g., an extract from a culture of Faecalibacterium prausnitzii, or a composition comprising optionally dried live cell or cell components and/or supernatant from a Faecalibacterium prausnitzii culture (FPS).

### BACKGROUND

Faecalibacterium prausnitzii is a commensal bacterium naturally occurring in the gastrointestinal tract of birds and mammals. WO2013130624A2, incorporated herein by reference, describes methods of using Faecalibacterium prausnitzii to improve weight gain, provide prophylaxis against diarrhea and improve feed efficiency in animals. WO2018118783A1, incorporated herein by reference, describes methods of using Faecalibacterium prausnitzii to improve milk production in animals, e.g., cattle.

Chicken is a low-cost animal-based protein source, and Americans eat more chicken than any other meat, with the average American eating 50.1 kg of chicken per year as of 2019. While consumption of beef and pork per capita has declined or remained steady over the last fifty years, the consumption of poultry has doubled. The largest cost in chicken production is feed, and improvements in the efficiency of feed utilization can reduce the cost to producers and ultimately to consumers. Reducing the amount of feed in poultry production benefits the environment and will lead to more sustainable meat production. We estimate that US poultry producers used approximately 40 billion kilograms of poultry feed in 2020. Improving the efficiency by which poultry convert their feed to meat can have a tremendous impact on poultry production and sustainability. If a commercially available product for poultry improves feed conversion ratio (FCR, sometimes referred to as feed conversion rate) by even 1%, then 400 million kilograms of feed could be saved every year in the US alone.

FCR is a measure of the efficiency of feed utilization in domestic livestock, including poultry (e.g., chickens, turkeys, ducks, geese, and guinea fowl). It is the ratio of inputs (feed) to outputs (e.g., weight gained or eggs produced). For broiler chickens, for example, a FCR of 1.6 means that for every 1.6 kg of feed consumed, the chickens gain 1 kilogram of weight. In the United States, commercial broilers typically weigh about 2.5 kg at 39 days of age, with a live-weight FCR on the order of 1.6 kg of feed per kilogram of body weight gain. Laying hens in commercial flocks typically produce about 330 eggs per year with a FCR of about 2 kg of feed per kilogram of eggs produced. The lower the FCR, the more efficient the birds are at converting feed into egg production, meat, and other consumable poultry products.

FCR depends on many factors, including the genetics of the birds, the health of the birds, the quality of the feed, and the temperature and other environmental factors. While FCR in poultry has improved greatly over the last century, the rate of improvement has slowed, and particularly in the United States and Canada, farmers have increasingly relied on antibiotics such as tetracycline, bacitracin, tylosin, salinomycin, virginiamycin and bambermycin as feed additives, to enhance FCR and reduce mortality. These antibiotics can reduce diseases such as necrotic enteritis caused by Clostridium perfringens. Even at sub-therapeutic doses, they can modify microbial diversity and relative abundance of different bacteria in the intestine, thereby impacting livestock feed utilization and growth by modifying the intestinal microbiome. Widespread use of antibiotics in poultry, however, has led to concerns about potential development of resistant bacteria and risks posed by antibiotic residues entering the human food supply and the environment.

Antibiotics and modern farming practices can affect the development of the microbiota, and subsequently affect digestion efficiency and susceptibility to pathogens. For example, broiler eggs are typically removed from their mother hen and hatched in hatcheries. In this clean and relatively sterile environment the hatched chicks do not acquire their microbiome in the same manner as they would have naturally. As microbiomes play a key role in protection from pathogen colonization and infection, birds with these altered microbiomes can be prone to dysbiosis and infection.

Despite the increased use of antibiotics, the contamination rate in poultry products such as meat and eggs from pathogens such as Campylobacter spp., pathogenic E. coli strains, and Salmonella spp. has risen. While Salmonella and Campylobacter appear to have little or no direct impact on bird performance, they present significant public health concerns. Campylobacter is a very common cause of food-borne diarrheal diseases in humans, certain strains of Escherichia coli, for example E. coli O157:H7, can cause serious diseases in humans, and Salmonella enterica is recognized as one of the world’s most common causes of human diarrhea. One cause of the increased incidence of enteric pathogens in commercial flocks may be the sterile environment found in hatcheries preventing the colonization and subsequent development of a healthy microbiota resulting in an opportunity for colonization and infections by opportunistic pathogens. Products that prevent the growth of these pathogens and allow development of a healthy gut microbiota would be of great value to the poultry industry and improve food safety.

There is a pressing need to increase poultry numbers, productivity, and ultimately protein production by poultry without intensifying the environmental footprint of agriculture, promoting antibiotic resistance, and increasing costs to the farmer. Regulatory constraints, including restrictions on the use of human medically important subtherapeutic antibiotics imposed on poultry producers continue to increase. Farmers are under pressure to identify alternative and cost-effective production strategies to improve feed efficiency while minimizing infectious pathogens in poultry products. There is a need for improved methods for sustainably and ethically increasing growth and production efficiency and health in poultry, reducing the risk of contamination with pathogens in poultry products, and reducing reliance on conventional antibiotics in poultry production.

### SUMMARY

It is now surprisingly discovered that Faecalibacterium prausnitzii and compositions made from culture of Faecalibacterium prausnitzii are useful in poultry production to improve meat quality (e.g., pH, drip loss, and color) and productivity by improving feed efficiency and health of the animals, as well as to improve the safety of end products derived from poultry by reducing colonization of poultry with human pathogens such as Salmonella and Campylobacter.

For example, FPS has been discovered to inhibit C. Perfringens, Salmonella and Campylobacter spp., in a dose-dependent manner.

Moreover, adding FPS to poultry feed is found to reduce FCR, thereby reducing protein production costs significantly, while simultaneously reducing bacterial load from poultry-related human pathogens.

The disclosure therefore provides, in a first embodiment, a method for improving feed efficiency in poultry, comprising administering an effective amount of FPS to the poultry.

In another embodiment, the disclosure provides a method for creating a gastrointestinal environment that is conducive to increasing the levels of commensal microbes, comprising administering an effective amount of FPS to the poultry.

In another embodiment, the disclosure provides a method for reducing pathogen load in poultry and/or reducing pathogen contamination of meat or eggs from poultry, comprising administering an effective amount of FPS to the poultry.

The disclosure provides, in another embodiment, a poultry food composition, hydrogel spray, water supplement, or composition for injection into eggs, comprising optionally dried cell components and supernatant from a Faecalibacterium prausnitzii culture, e.g., wherein the Faecalibacterium prausnitzii cell components and supernatant are dried, e.g. lyophilized.

The disclosure provides, in another embodiment, a method of making a poultry food composition comprising optionally dried cell components and supernatant from a Faecalibacterium prausnitzii culture, the method comprising culturing a strain of Faecalibacterium prausnitzii, centrifuging the Faecalibacterium culture, to separate it into a supernatant portion and a sediment portion, drying the product, and combining it with poultry feed; for example, comprising the following steps:
a. Culturing Faecalibacterium prausnitzii;
b. Optionally killing the Faecalibacterium prausnitzii, e.g., by exposing to oxygen;
c. Centrifuging the optionally killed Faecalibacterium prausnitzii culture, to separate it into a supernatant portion and a sediment portion;
d. Removing excess water from the supernatant portion, e.g., using reverse osmosis
e. Combining the product of step (d) with the sediment portion;
f. Drying the product of step (e) to obtain a powder, e.g., by lyophilization;
g. Admixing the powder thus produced with poultry feed.

Further areas of applicability of the present disclosure become apparent from the detailed description provided hereinafter. It should be understood that the detailed description and specific examples, while indicating the preferred embodiment of the invention, are intended for purposes of illustration only and are not intended to limit the scope of the invention.

### DESCRIPTION OF FIGURES

FIG. 1 depicts in vitro growth inhibition of S. enterica with four strains of Faecalibacterium prausnitzii.

FIG. 2 depicts effects of FPS on growth of broiler chickens at six weeks of age. *p < 0.05.

### DESCRIPTION

In a first embodiment, the disclosure provides a method (Method 1) for improving feed efficiency and/or meat or egg quality in poultry, comprising administering to the poultry an effective amount of Faecalibacterium prausnitzii or compositions made therefrom, e.g., an extract from a culture of Faecalibacterium prausnitzii, or a composition comprising optionally dried live cell or cell components and/or supernatant from a Faecalibacterium prausnitzii culture (FPS), for example:

1.1. Method 1, wherein the poultry are selected from chickens, turkeys, ducks, geese, and guinea fowl.

1.2. Any foregoing method wherein the poultry are chickens.

1.3. Any foregoing method wherein the poultry are intended for meat production, e.g., broiler chickens.

1.4. Any foregoing method wherein the poultry are intended for egg production, e.g., laying hens.

1.5. Any foregoing method wherein the feed efficiency is measured by feed conversion ratio (FCR).

1.6. Any foregoing method wherein the poultry exhibit an improvement in feed conversion ratio (FCR), e.g., an improvement of at least 0.5%, relative to control poultry, which do not receive FPS.

1.7. Any foregoing method wherein the poultry have higher breast weight and breast muscle weight relative to control poultry which do not receive FPS.

1.8. Any foregoing method wherein the Faecalibacterium prausnitzii is cultured in media free of any animal derived components comprising optimized mixture of nitrogen and carbon sources, and other nutritional components, including peptides, amino acids, carbohydrates, minerals, vitamins, and salts.

1.9. Any foregoing method wherein the Faecalibacterium prausnitzii is a strain that is not naturally found in poultry, e.g. a strain of bovine origin.

1.10. Any foregoing method wherein the Faecalibacterium prausnitzii has a 16 S rDNA sequence comprising a sequence selected from GenBank (NCBI) accession numbers KJ957841 to KJ957877.

1.11. Any foregoing method wherein the Faecalibacterium prausnitzii is a strain that exhibits elevated production of butyrate, e.g. relative to a control strain, e.g., relative to reference strain DSM 17677.

1.12. Any foregoing method wherein the poultry do not receive antibiotics, e.g., antibiotics selected from one or more of tetracycline, bacitracin, tylosin, salinomycin, virginiamycin and bambermycin.

1.13. Any foregoing method wherein the method comprises a method of improving meat quality as measured by pH, drip loss, and color of the meat.

1.14. Any foregoing method wherein the poultry receive live Faecalibacterium prausnitzii.

1.15. Any foregoing method wherein the poultry receive a composition comprising optionally dried cell components and supernatant from a Faecalibacterium prausnitzii culture, e.g., a composition according to Composition 1, infra.

1.16. Any foregoing method wherein the administration of effective amount of Faecalibacterium prausnitzii or compositions made therefrom is by feeding the poultry a composition comprising optionally dried cell components and supernatant from a Faecalibacterium prausnitzii culture, or by spraying chicks with a gel comprising a composition comprising optionally dried cell components and supernatant from a Faecalibacterium prausnitzii culture, or by injecting eggs with a composition comprising optionally dried cell components and supernatant from a Faecalibacterium prausnitzii culture.

1.17. Any foregoing method wherein the poultry receive an extract from a culture of Faecalibacterium prausnitzii.

1.18. Any foregoing method wherein the poultry receive a poultry food composition or composition adapted for delivery to poultry comprising cell components and supernatant from a Faecalibacterium prausnitzii culture.

1.19. Any foregoing method wherein the poultry receive a poultry food composition or composition adapted for delivery to poultry comprising cell components and supernatant from a Faecalibacterium prausnitzii culture, wherein the Faecalibacterium prausnitzii cell components and supernatant are dried.

1.20. Any foregoing method wherein the poultry receive a poultry food composition or composition adapted for delivery to poultry comprising cell components and supernatant from a Faecalibacterium prausnitzii culture, wherein the Faecalibacterium prausnitzii cell components and supernatant are dried and in the form of particles having an average diameter of less than 100 microns.

1.21. Any foregoing method wherein the poultry receive a poultry food composition or composition adapted for delivery to poultry comprising cell components and supernatant from a Faecalibacterium prausnitzii culture, wherein the Faecalibacterium prausnitzii cell components and supernatant have a concentration of 50 - 500 ppm, preferably 80 - 250 ppm.

1.22. Any foregoing method wherein the poultry receive a poultry feed comprising cell components and supernatant from a Faecalibacterium prausnitzii culture, wherein the feed is optimized to meet the nutritional requirements of poultry and comprises cereal grains, oilseed meal, animal by-product meal, fats, and vitamin and mineral premixes.

1.23. Any foregoing method wherein the poultry are chicks which are sprayed with a gel spray, e.g., a hydrogel spray, comprising cell components and supernatant from a Faecalibacterium prausnitzii culture.

1.24. Any foregoing method wherein the poultry receive a poultry food composition or composition adapted for delivery to poultry comprising cell components and supernatant from a Faecalibacterium prausnitzii culture, which is a poultry food supplement.

1.25. Any foregoing method wherein the poultry receive a poultry food composition or composition adapted for delivery to poultry comprising cell components and supernatant from a Faecalibacterium prausnitzii culture, which is a solution, suspension or powder for admixture with drinking water.

1.26. Any foregoing method wherein the poultry receive a poultry food composition or composition adapted for delivery to poultry comprising cell components and supernatant from a Faecalibacterium prausnitzii culture, which is a composition for injection into eggs.

1.27. Any foregoing method wherein the poultry receive FPS during the first week of life.

1.28. Any foregoing method wherein the poultry receive FPS during the first 15 days of life.

1.29. Any foregoing method wherein the poultry receive FPS during the first 30-60 days of life.

1.30. Any foregoing method wherein the poultry receive FPS during the first 60 days of life, e.g., only during the first 30 days of life, e.g., the first week of life, but the improvement in feed efficiency is sustained throughout the bird’s life.

1.31. Any foregoing method wherein the poultry are intended for meat consumption, e.g., wherein the poultry are broiler chickens or turkeys, and receive FPS during the first 60 days of life, e.g., the first 30 days of life, e.g., the first week of life, and also receive FPS during a finishing period prior to slaughter.

1.32. Any foregoing method wherein the poultry are broiler chickens that receive FPS in feed for their first week of life and wherein the chickens receiving FPS exhibit significantly higher weight at six weeks than chickens which do not receive FPS, e.g., wherein the breast weight and breast muscle weight are significantly higher in FPS treated birds in comparison to untreated birds.

1.33. Any foregoing method wherein the poultry are broiler chickens that receive FPS in feed for their first week of life, e.g., during the first 15 days of life, and wherein the chickens receiving FPS exhibit significantly higher weight and lower FCR at 28 days than chickens which do not receive FPS,

1.34. Any foregoing method wherein the poultry are broiler chickens that receive FPS in feed for their first week of life, e.g., during the first 15 days of life, and wherein the chickens receiving FPS exhibit significantly higher weight and lower FCR at 42 days than chickens which do not receive FPS,

1.35. Any foregoing method wherein the poultry are fed with feed sprayed with a composition comprising dried cell components and supernatant from a Faecalibacterium prausnitzii culture (FPS-1) at a concentration of 100-1000 ppm, e.g., about 250 ppm, for the first week of life, e.g., for the first 15 days of life.

1.36. Any foregoing method wherein the poultry display enhanced enterocyte proliferation and localization during early life, e.g., during the first week or the first 15 days of life, resulting in increased surface area (due to higher intestinal villa), increased ability to absorb nutrients, and accelerated growth, compared to poultry which does not receive FPS.

1.37. Any foregoing method wherein the poultry receive FPS during their entire life.

The disclosure provides in another embodiment, the disclosure provides a method (Method 2) for reducing morbidity and/or mortality in poultry from gastrointestinal disease and/or promoting healthy gut microbiota in poultry, comprising administering to the poultry an effective amount Faecalibacterium prausnitzii or compositions made therefrom, e.g., an extract from a culture of Faecalibacterium prausnitzii, or a composition comprising optionally dried live cell or cell components and/or supernatant from a Faecalibacterium prausnitzii culture (FPS), for example:

2.1.Method 2, wherein the poultry are selected from chickens, turkeys, ducks, geese, and guinea fowl.

2.2. Any foregoing method wherein the poultry are chickens.

2.3. Any foregoing method wherein the poultry are intended for meat production, e.g., broiler chickens.

2.4. Any foregoing method wherein the poultry are intended for egg production, e.g., laying hens.

2.5. Any foregoing method wherein the gastrointestinal disease is an infectious disease.

2.6. Any foregoing method wherein the gastrointestinal disease is a bacterial infection, e.g. selected from one or more of (i) necrotic enteritis, e.g., caused by Clostridium perfringens; (ii) Campylobacteriosis, e.g., caused by C. jejuni, C. coli and/or C. lardis; and (iii) salmonellosis (pullorum or bacillary white diarrhea), e.g., caused by S. pullorum.

2.7. Any foregoing method wherein the gastrointestinal disease is a protozoal infection, e.g., Coccidiosis, e.g., caused by Eimeria spp.

2.8. Any foregoing method wherein the poultry exhibit an improvement in feed conversion ratio (FCR), e.g., an improvement of at least 0.5%, relative to control poultry, which do not receive the FPS.

2.9. Any foregoing method wherein the Faecalibacterium prausnitzii is cultured in media free of any animal derived components comprising optimized mixture of nitrogen and carbon sources, and other nutritional components, including peptides, amino acids, carbohydrates, minerals, vitamins, and salts.

2.10. Any foregoing method wherein the Faecalibacterium prausnitzii is a strain that is not naturally found in poultry, e.g. a strain of bovine origin.

2.11. Any foregoing method wherein the Faecalibacterium prausnitzii has a 16 S rDNA sequence comprising a sequence selected from GenBank (NCBI) accession numbers KJ957841 to KJ957877.

2.12. Any foregoing method wherein the Faecalibacterium prausnitzii is a strain that exhibits elevated production of butyrate, e.g. relative to a control strain, e.g., relative to reference strain DSM 17677.

2.13. Any foregoing method wherein the poultry do not receive antibiotics, e.g., antibiotics selected from one or more of tetracycline, bacitracin, tylosin, salinomycin, virginiamycin and bambermycin.

2.14. Any foregoing method wherein the poultry receive live Faecalibacterium prausnitzii.

2.15. Any foregoing method wherein the poultry receive a composition comprising optionally dried cell components and supernatant from a Faecalibacterium prausnitzii culture, e.g., a composition according to Composition 1, infra.

2.16. Any foregoing method wherein the administration of effective amount of Faecalibacterium prausnitzii or compositions made therefrom is by feeding the poultry a composition comprising optionally dried cell components and supernatant from a Faecalibacterium prausnitzii culture, or by spraying chicks with a gel comprising a composition comprising optionally dried cell components and supernatant from a Faecalibacterium prausnitzii culture, or by injecting eggs with a composition comprising optionally dried cell components and supernatant from a Faecalibacterium prausnitzii culture.

2.17. Any foregoing method wherein the poultry receive an extract from a culture of Faecalibacterium prausnitzii.

2.18. Any foregoing method wherein the poultry receive a poultry food composition or composition adapted for delivery to poultry comprising cell components and supernatant from a Faecalibacterium prausnitzii culture.

2.19. Any foregoing method wherein the poultry receive a poultry food composition or composition adapted for delivery to poultry comprising cell components and supernatant from a Faecalibacterium prausnitzii culture, wherein the Faecalibacterium prausnitzii cell components and supernatant are dried.

2.20. Any foregoing method wherein the poultry receive a poultry food composition or composition adapted for delivery to poultry comprising cell components and supernatant from a Faecalibacterium prausnitzii culture, wherein the Faecalibacterium prausnitzii cell components and supernatant are dried and in the form of particles having an average diameter of less than 100 microns.

2.21. Any foregoing method wherein the poultry receive a poultry feed comprising cell components and supernatant from a Faecalibacterium prausnitzii culture, wherein the feed is optimized to meet the nutritional requirements of poultry and comprises cereal grains, oilseed meal, animal by-product meal, fats, and vitamin and mineral premixes.

2.22. Any foregoing method wherein the poultry are chicks which are sprayed with a gel spray, e.g., a hydrogel spray, comprising cell components and supernatant from a Faecalibacterium prausnitzii culture.

2.23. Any foregoing method wherein the poultry receive a poultry food composition or composition adapted for delivery to poultry comprising cell components and supernatant from a Faecalibacterium prausnitzii culture, which is a poultry food supplement.

2.24. Any foregoing method wherein the poultry receive a poultry food composition or composition adapted for delivery to poultry comprising cell components and supernatant from a Faecalibacterium prausnitzii culture, which is a solution, suspension or powder for admixture with drinking water.

2.25. Any foregoing method wherein the poultry receive a poultry food composition or composition adapted for delivery to poultry comprising cell components and supernatant from a Faecalibacterium prausnitzii culture, which is a composition for injection into eggs.

2.26. Any foregoing method wherein the poultry are fed with feed sprayed with a composition comprising dried cell components and supernatant from a Faecalibacterium prausnitzii culture (FPS-1) at a concentration of 100-1000 ppm, e.g., about 250 ppm, for the first week of life, e.g., for the first 15 days of life.

2.27. Any foregoing method wherein FPS improves the gut health of the poultry relative to untreated poultry, e.g., wherein the treated poultry exhibit enhanced bioproduction or secretion of mucin and/or enhanced tight junctions in the gut, thereby reducing the susceptibility of the poultry to infection by pathogens.

The disclosure provides in another embodiment, the disclosure provides a method (Method 3) for reducing pathogen load in poultry and/or reducing pathogen contamination of meat or eggs from poultry, comprising administering to the poultry an effective amount of Faecalibacterium prausnitzii or compositions made therefrom, e.g., an extract from a culture of Faecalibacterium prausnitzii, or a composition comprising optionally dried live cell or cell components and/or supernatant from a Faecalibacterium prausnitzii culture (FPS), for example:

3.1. Method 3, wherein the poultry are selected from chickens, turkeys, ducks, geese, and guinea fowl.

3.2. Any foregoing method wherein the poultry are chickens.

3.3. Any foregoing method wherein the poultry are intended for meat production, e.g., broiler chickens.

3.4. Any foregoing method wherein the poultry are intended for egg production, e.g., laying hens.

3.5. Any foregoing method wherein the pathogen is a human pathogen, e.g. selected from Campylobacter spp. (e.g., C. jejuni), pathogenic E. coli strains (e.g., E. coli 0157:H7), Enterobacteriaceae spp. and Salmonella spp. (e.g., S. enterica).

3.6. Any foregoing method wherein the Faecalibacterium prausnitzii is cultured in media free of any animal derived components comprising optimized mixture of nitrogen and carbon sources, and other nutritional components, including peptides, amino acids, carbohydrates, minerals, vitamins, and salts.

3.7. Any foregoing method wherein the Faecalibacterium prausnitzii is a strain that is not naturally found in poultry, e.g. a strain of bovine origin.

3.8. Any foregoing method wherein the Faecalibacteriumprausnitzii has a 16 S rDNA sequence comprising a sequence selected from GenBank (NCBI) accession numbers KJ957841 to KJ957877.

3.9. Any foregoing method wherein the Faecalibacterium prausnitzii is a strain that exhibits elevated production of butyrate, e.g. relative to a control strain, e.g., relative to reference strain DSM 17677.

3.10. Any foregoing method wherein the poultry do not receive antibiotics, e.g., antibiotics selected from one or more of tetracycline, bacitracin, tylosin, salinomycin, virginiamycin and bambermycin.

3.11. Any foregoing method wherein the poultry receive live Faecalibacterium prausnitzii.

3.12. Any foregoing method wherein the poultry receive a composition comprising optionally dried cell components and supernatant from a Faecalibacterium prausnitzii culture, e.g., a composition according to Composition 1, infra.

3.13. Any foregoing method wherein the poultry receive an extract from a culture of Faecalibacterium prausnitzii.

3.14. Any foregoing method wherein the administration of effective amount of Faecalibacterium prausnitzii or compositions made therefrom is by feeding the poultry a composition comprising optionally dried cell components and supernatant from a Faecalibacterium prausnitzii culture, or by spraying chicks with a gel comprising a composition comprising optionally dried cell components and supernatant from a Faecalibacterium prausnitzii culture, or by injecting eggs with a composition comprising optionally dried cell components and supernatant from a Faecalibacterium prausnitzii culture.

3.15. Any foregoing method wherein the poultry receive a poultry food composition or composition adapted for delivery to poultry comprising cell components and supernatant from a Faecalibacterium prausnitzii culture.

3.16. Any foregoing method wherein the poultry receive a poultry food composition or composition adapted for delivery to poultry comprising cell components and supernatant from a Faecalibacterium prausnitzii culture, wherein the Faecalibacterium prausnitzii cell components and supernatant are dried.

3.17. Any foregoing method wherein the poultry receive a poultry food composition or composition adapted for delivery to poultry comprising cell components and supernatant from a Faecalibacterium prausnitzii culture, wherein the Faecalibacterium prausnitzii cell components and supernatant are dried and in the form of particles having an average diameter of less than 100 microns.

3.18. Any foregoing method wherein the poultry receive a poultry feed comprising cell components and supernatant from a Faecalibacterium prausnitzii culture, wherein the feed is optimized to meet the nutritional requirements of poultry and comprises cereal grains, oilseed meal, animal by-product meal, fats, and vitamin and mineral premixes.

3.19. Any foregoing method wherein the poultry are chicks which are sprayed with a gel spray, e.g., a hydrogel spray, comprising cell components and supernatant from a Faecalibacterium prausnitzii culture.

3.20. Any foregoing method wherein the poultry receive a poultry food composition or composition adapted for delivery to poultry comprising cell components and supernatant from a Faecalibacterium prausnitzii culture, which is a poultry food supplement.

3.21. Any foregoing method wherein the poultry receive a poultry food composition or composition adapted for delivery to poultry comprising cell components and supernatant from a Faecalibacterium prausnitzii culture, which is a solution, suspension or powder for admixture with drinking water.

3.22. Any foregoing method wherein the poultry receive a poultry food composition or composition adapted for delivery to poultry comprising cell components and supernatant from a Faecalibacterium prausnitzii culture, which is a composition for injection into eggs.

3.23. Any foregoing method wherein the poultry are fed with feed sprayed with a composition comprising dried cell components and supernatant from a Faecalibacterium prausnitzii culture (FPS-1) at a concentration of 100-1000 ppm, e.g., about 250 ppm, for the first week of life, e.g., for the first 15 days of life.

3.24. Any foregoing method wherein FPS improves the gut health of the poultry relative to untreated poultry, e.g., wherein the treated poultry exhibit enhanced bioproduction or secretion of mucin and/or enhanced tight junctions in the gut, thereby reducing the susceptibility of the poultry to infection by pathogens.

The disclosure provides, in another embodiment, a poultry food composition or composition adapted for delivery to poultry (Composition 1) comprising optionally dried cell components and/or supernatant or derivatives therefrom from a Faecalibacterium prausnitzii culture, e.g.:

1.1. Composition 1 wherein a Faecalibacterium prausnitzii culture has been centrifuged to separate it into a supernatant portion and a sediment portion, which are then recombined.

1.2. Any foregoing composition wherein the Faecalibacterium prausnitzii culture is killed, e.g., by exposure to oxygen, then centrifuged to separate it into a supernatant portion and a sediment portion.

1.3.Any foregoing composition wherein the Faecalibacterium prausnitzii cell components and supernatant are dried.

1.4. Any foregoing composition wherein the Faecalibacterium prausnitzii killed cell components and supernatant are lyophilized.

1.5. Any foregoing composition which is suitable for administration as a food additive to poultry food.

1.6. Any foregoing composition which is a poultry feed, comprising optionally dried cell components and supernatant from a Faecalibacterium prausnitzii culture.

1.7. Any foregoing composition which is a poultry feed comprising cereal grains (e.g., selected from wheat, barley, triticale, maize, sorghum, and combinations thereof), oilseed meal (e.g., produced from soybean, canola, field peas, and combinations thereof), animal by-product meal (e.g., selected from fish meal, meat meal, bone meal, and combinations thereof), fats (e.g., selected from fish oils, vegetable oils, and combinations thereof), and vitamin and mineral premixes, wherein the poultry feed is optimized to meet the nutritional requirements of poultry.

1.8. Any foregoing method wherein the Faecalibacterium prausnitzii is a strain that is not naturally found in poultry, e.g. a strain of bovine origin.

1.9.Any foregoing composition wherein the Faecalibacterium prausnitzii is a strain that exhibits elevated production of butyrate, e.g. relative to a control strain, e.g., relative to reference strain DSM 17677.

1.10. Any foregoing composition wherein the Faecalibacterium prausnitzii is cultured in media free of any animal derived components comprising optimized mixture of nitrogen and carbon sources, and other nutritional components, including peptides, amino acids, carbohydrates, minerals, vitamins, and salts.

1.11. Any foregoing composition wherein the Faecalibacterium prausnitzii has a 16 S rDNA sequence comprising a sequence selected from GenBank (NCBI) accession numbers KJ957841 to KJ957877.

1.12. Any foregoing composition wherein the composition is in the form of a hydrogel, e.g., comprising water, one or more gelling agents (e.g., selected from carboxymethylcellulose, hydroxypropylmethyl cellulose, methyl cellulose, alginate, pectin, carrageenan, gellan, gelatin, agar, modified starch, xanthan gum, locust bean gum, and combinations thereof), and the cell components and supernatant or derivatives therefrom from a Faecalibacterium prausnitzii culture, and optionally further comprising vitamins, minerals and nutrients, e.g., comprising ingredients selected from one or more of the group consisting of vegetable oil, molasses, ascorbic acid, vitamin blend (e.g., comprising one or more of vitamin A palmitate, vitamin D3, vitamin E acetate, niacin, riboflavin, calcium pantothenate, pyridoxine HC1, and thiamine HCl), choline bitartrate, corn fiber, wheat protein isolate, electrolyte blend (e.g., sodium chloride and potassium chloride), and mineral blends (e.g., comprising one or more of potassium phosphate dibasic, calcium carbonate, sodium phosphate, calcium phosphate, sodium chloride, magnesium sulfate, ferric citrate, manganese sulfate, potassium iodide, zinc carbonate, and cupric sulfate).

1.13. Any foregoing composition which is adapted to be added to drinking water for the poultry.

1.14. Any foregoing composition which is adapted to be injected in ovo.

1.15. Any foregoing composition which is adapted to be sprayed on chicks, e.g., in a gel spray.

1.16. Any foregoing composition wherein the cell components and supernatant from a Faecalibacterium prausnitzii culture are present in a concentration effective in any of Methods 1, 2, or 3, supra, e.g., in a concentration effective to enhance the feed conversion ratio of the poultry, measured with respect to weight gain, meat production, or egg production.

1.17. Any foregoing composition wherein the cell components and supernatant from a Faecalibacterium prausnitzii culture are present in a concentration of 50 - 500 ppm, preferably 80 - 250 ppm.

1.18. Any foregoing composition comprising dried cell components and supernatant or derivatives therefrom from a Faecalibacterium prausnitzii culture, in the form of particles having an average diameter of less than 100 microns.

1.19. Any foregoing composition which is obtained or obtainable by the steps of:
a. Culturing Faecalibacterium prausnitzii;
b. Optionally killing the Faecalibacterium prausnitzii, e.g., by exposing to oxygen;
c. Centrifuging the optionally killed Faecalibacterium prausnitzii culture, to separate it into a supernatant portion and a sediment portion;
d. Removing excess water from the supernatant portion, e.g., using reverse osmosis;
e. Combining the product of step (d) with the sediment portion;
f. Drying the product of step (e) to obtain a powder, e.g., using lyophilization; and
g. Optionally combining the powder thus produced with a poultry feed or delivery vehicle (e.g., water, gel, or supplement) suitable for administration to poultry.

1.20. Any foregoing composition which is obtained or obtainable by the steps of:
a. Culturing Faecalibacterium prausnitzii;
b. Killing the Faecalibacterium prausnitzii by exposing to oxygen;
c. Centrifuging the killed Faecalibacterium prausnitzii culture, to separate it into a supernatant portion and a sediment portion;
d. Removing excess water from the supernatant portion using reverse osmosis;
e. Combining the product of step (d) with the sediment portion;
f. Drying the product of step (e) to obtain a powder using lyophilization; and
g. Combining the powder thus produced with a poultry feed or delivery vehicle (e.g., water, gel or supplement) suitable for administration to poultry.

The disclosure further provides Faecalibacterium prausnitzii, or a composition made from a culture of Faecalibacterium prausnitzii, e.g., an extract from a culture of Faecalibacterium prausnitzii, or a composition comprising Faecalibacterium prausnitzii cell components that may also include supernatant and derivatives therefrom, e.g. a composition according to any of Composition 1, et seq., for use in accordance with any of Method 1, et seq., Method 2, et seq., or Method 3, et seq.

In some embodiments, the methods and compositions herein use live Faecalibacterium prausnitzii cells that may also include supernatant and derivatives therefrom. In some embodiments, the methods and compositions herein use killed Faecalibacterium prausnitzii cells that may also include supernatant and derivatives therefrom. In some embodiments, the methods and compositions herein use supernatant and derivatives therefrom of Faecalibacterium prausnitzii. In some embodiments, the methods and compositions herein use killed cells and supernatant of Faecalibacterium prausnitzii.

In some embodiments, the strains of Faecalibacterium prausnitzii used exhibit relatively high butyrate production, e.g., as measured using gas chromatography of culture supernatant indicating a concentration of butyrate exceeding 1000 ppm. For example, Faecalibacterium prausnitzii isolates and a reference strain, DSM 17677, are inoculated in 25 ml of nutrient broth and incubated at 37° C. for 48 h under anaerobic conditions. The culture is centrifuged, the supernatant is collected, and the concentration of acetate, butyrate, propionate and isobutyrate in the media before inoculation and in the supernatant of the culture was measured by gas chromatography. Samples are injected into a gas chromatograph, and the analysis is performed according to the manufacturer’s protocol. Isolates producing relatively high levels of butyrate in the supernatant, e.g., greater than the reference strain, e.g., at least 1000 ppm, are selected.

In a further embodiment, the disclosure provides a method of making a poultry food composition or composition adapted for delivery to poultry comprising cell components and supernatant from a Faecalibacterium prausnitzii culture, e.g., a composition according to Composition 1, et seq., comprising culturing Faecalibacterium prausnitzii, centrifuging the Faecalibacterium culture to separate it into a supernatant portion and a sediment portion, drying the product, and combining it with a poultry feed or delivery vehicle, e.g., comprising the following steps:
a. Culturing the Faecalibacterium;
b. Optionally killing the Faecalibacterium, e.g., by exposing to oxygen;
c. Centrifuging the optionally killed Faecalibacterium prausnitzii culture, to separate it into a supernatant portion and a sediment portion;
d. Removing excess water from the supernatant portion, e.g., using reverse osmosis and/or using drying, e.g., spray-drying or evaporation;
e. Combining the product of step (d) with the sediment portion;
f. Drying, e.g., lyophilizing, the product of step (e) to obtain a powder;
g. Admixing the powder thus produced with poultry feed or delivery vehicle selected from water, gel and feed supplement suitable for consumption by poultry.

### Example 1: Inhibition of Pathogens Using FPS

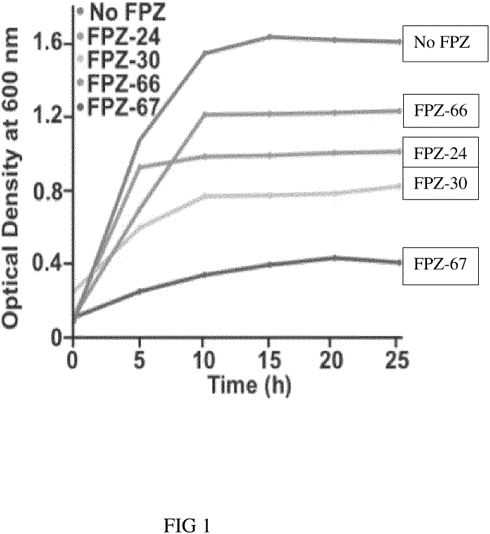

In vitro growth inhibition of S. enterica with supernatants from Faecalibacterium prausnitzii strains are depicted in FIG. 1. Supernatant from cultures of four different strains (FPZ-24, -30, -66 and -67) is incubated with Salmonella enterica SE12 for 24 h. Faecalibacterium prausnitzii growth media is used as a control. As depicted in FIG. 1, the supernatant from each of the four strains of Faecalibacterium prausnitzii shows dose dependent inhibition of S. enterica growth.

The antimicrobial properties of Faecalibacterium prausnitzii supernatant are further characterized against strains of Salmonella enterica and Campylobacter jejuni. Assays including well diffusion assay, agar spot assay, microtiter assay and plate dilution methods are employed to identify the products that exert highest antimicrobial effect against S. enterica and Campylobacter jejuni. In addition, the ability of antimicrobial products to inhibit mucin and collagen binding and biofilm formation are investigated. A primary chicken intestinal cell line is used in the colonization assay. Experiments include identification of the sub inhibitory concentration of the test products, pathogen attachment, invasion and translocation assays, testing for cell cytotoxicity, toxin production, inflammatory response and gene expression assays.

Bacterial Strains and Growth Conditions: Four strains (chicken isolates) of Salmonella Enteritidis (SE; SE-12, SE-22, SE-28 and SE-31) are used in the study. All bacteriological media used in the study are procured from Difco (Difco Becton, MD, USA). SE are grown in Tryptic Soya broth (TSB) at 37oC overnight. After incubation, the cultures are centrifuged (3000Xg, 12 min, 4° C.), and washed twice in phosphate buffered saline (PBS, pH 7.0), separately. The pellet is resuspended in PBS and used as the inoculum. Bacterial counts in the Salmonella cultures are confirmed following serial dilution and plating on Tryptic Soya agar (TSA).

Faecalibacterium prausnitzii supernatant screening for anti-Salmonella activity: Salmonella strains grown overnight are subcultured for 2 h in TSB. Supernatants from the Faecalibacterium prausnitzii strains are added in increasing concentration to the Salmonella culture (~6 log CFU/ml) in an early exponential phase and incubated further for 24 h at 37° C. Salmonella viability is assayed at specified times during the incubation by plating on TSA (Das et al., 2013). Duplicate wells are used for each treatment and control, and the experiment is repeated three times.

Estimation of sub-inhibitory concentration (SIC) of Faecalibacterium prausnitzii supernatant: Approximately 6 log CFU/ml of Salmonella are inoculated into TSB supplemented with different concentrations of Faecalibacterium prausnitzii supernatant and incubated at 37° C. for 24 h. Following incubation, the surviving Salmonella population are enumerated by dilution and plating on TSA. The highest concentration of supernatant which does not inhibit Salmonella growth is determined to be the SIC.

Avian epithelial cell line: To evaluate the inhibitory effect of Faecalibacterium prausnitzii strains on Salmonella colonization in vitro, an avian epithelial cell line (BATC; Budgerigar abdominal tumor cells) is used. The monolayers are cultured in Dulbecco’s modified eagle medium (DMEM, Fisher Scientific) supplemented with 10% fetal calf serum (FCS, Invitrogen). Following 3 propagations, the cells are seeded into 24-well tissue culture plates containing whole medium (DMEM + 10% FBS) and incubated at 37° C. with 5% CO2 to reach a confluency of >95%.

Inhibition of Salmonella adhesion to BATC: Faecalibacterium prausnitzii supernatant is added to the BATC monolayer and incubated at 37° C. and 5% CO₂ for 24 h. The cells are then washed three times with PBS and infected with S. enterica (SE) at a multiplicity of infection (MOI) of 1:1 (BATC: Salmonella) for 2 h (Koo et al., 2013). The wells are washed three times with PBS, and the BATC is lysed using 0.1 % Triton X 100 in PBS and incubated at 37oC and 5% CO2 for 10 min to release the adherent and internalized Salmonella. The cell homogenates are diluted tenfold in PBS and plated on TSA to enumerate the BATC associated Salmonella population (Kollanoor-Johny et al., 2012a). The adhesion assay for each Salmonella strain is run in duplicate and replicated three times.

Inhibition of Salmonella invasion in BATC: For the internalization assay, BATC monolayers are pre-exposed to the different FPS supernatants and infected with SE as described previously. Following infection for 2 h, monolayers are washed three times with DMEM and incubated in whole media containing 100 µg/ml gentamicin for 1 h to kill all the adhered (extra cellular) bacteria. Internalized Salmonella are enumerated after triton lysis and plating on TSA. The invasion assay for each Salmonella strain is run in duplicate and replicated three times.

Inhibition of Salmonella invasion and survival in HTC: Chicken macrophage cells (HTC) are cultivated in RPMI 1640 containing 10% FBS and incubated at 37oC and 5% CO2 for 24 h. The cells are activated using 0.1 pg/ml phorbol myristate acetate (PMA). Following activation, inhibition of Salmonella invasion and survival in macrophages are assayed. Salmonella is grown in the presence or absence of the SICs of the FPS supernatants at 37° C. for 24 h. Following overnight growth, the cultures are washed and resuspended in RPMI 1640. For the internalization assay, activated and attached macrophages are infected with SE at an MOI of 1:1 and incubated at 37° C. and 5% CO₂ for 2 h. The unattached Salmonella are removed by washing with RPMI, and fresh media supplemented with 100 pg/ml gentamicin is added. HTC monolayers are incubated further for an additional 1 h to kill any Salmonella attached to the surface. The media in the wells are changed every day with whole media containing 10 µg/ml gentamicin. The cells are washed thrice with PBS, lysed at 2, 24, 48, and 72 h using 0.1% Triton X-100 before serial dilution and plating to enumerate the surviving intracellular Salmonella population. The assay for each Faecalibacterium prausnitzii supernatant and Salmonella strain is run in duplicate, and the entire experiment repeated three times.

Statistical analysis: Log values of Salmonella count at different time periods are tested for significance at a p value of <0.05 using PROC GLIMMIX procedure of SAS (version 9.2; SAS Institute Inc., Cary, NC). For motility, adhesion, invasion and RT-qPCR assays, the data are analyzed using the PROC-MIXED procedure of SAS. The results of the three independent tests are tested for significance at a p value of <0.05 using MANOVA.

### Example 2: Effect of FPS on Growth of Broiler Chickens

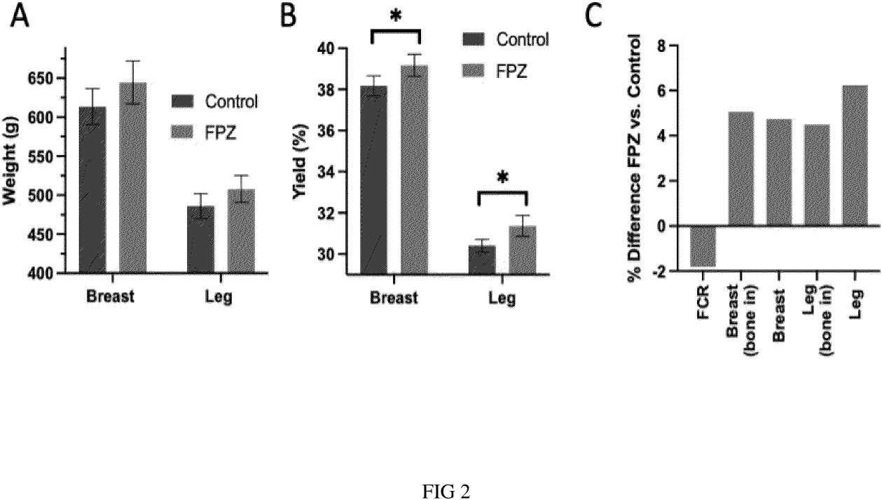

Diet effects of a composition comprising cell components and supernatant (FPS-1) from a Faecalibacterium prausnitzii culture on growth of broiler chickens at six weeks of age are depicted in FIG. 2. FIG. 2A shows differences in breast and leg weight, 2B shows differences in breast and leg meat yield, and 2C shows percent differences of FCR, breast and leg weight between control, and 80 mg FPS/kg feed, FPS treatment for 7 days.

Day old chicks are administered FPS-1 in feed for one week. After six weeks, chickens from each group are measured for live weight and FCR calculated. Birds from each group are sacrificed (control, n=41; FPS-treated n=42) and breast and leg weights (bone in and meat only) were calculated. These results are shown in FIG. 2 (control in left columns vs. FPS treated in right columns). Breast and leg weight and yield at six weeks are higher for the treated group (FIGS. 2A and 2B respectively) even though the treatment was only applied for the first week of life. For all weight measurements, FPS administration leads to higher weight compared to control birds. Breast weight and breast muscle weight in particular are significantly higher in FPS treated birds in comparison to untreated birds. FCR is also seen to decrease substantially, from 1.92 in non-treated controls to 1.74 in birds treated with FPS, indicating significantly greater feed efficiency. As noted above, even a small improvement in FCR results can have a significant economic benefit for poultry producers; these data show an improvement in FCR on the order of 9%. These results moreover show that the beneficial effects of the FPS are sustained well beyond the period of administration, and that the FPS can be administered in a way that is not disruptive to current processes and produces a large effect on growth performance and feed conversion efficiency.

### Trial B for FPS Effect on Growth of Broiler Chickens

In one trial scenario, 10 replicate pens of a group of 25 chickens represent each of three treatment groups. The treatment groups consist of an untreated control group, a group which receives a composition comprising cell components and supernatant (FPS-1) from a Faecalibacterium prausnitzii culture for 7 days, and a group which receives FPS-1 for 15 days.

On the day of hatch, chickens are weighed and assigned to one of these three groups. For the 7-day treatment, the chickens are fed with feed sprayed with FPS-1 at a concentration of 250 ppm for the first week of life. For the 15-day treatment, the chickens are fed with feed that is sprayed with FPS-1 at a concentration of 250 ppm for the first 15 days of life. At 15, 28, and 42 days after the day of hatch, the chickens from each group are measured for live weight and FCR is calculated. The intake of feed by the chickens in each of the three treatment groups is shown in Table 1 and was similar among the three groups.

TABLE 1

Feed Intake Comparison of Control, 7-day Treatment, and 15-day Treatment
Feed Intake (kg/pen)

| | | | |
|---|---|---|---|
| Age (days) | 15 | 28 | 42 |
| Control | 12.59 | 44.83 | 93.40 |
| 7- Day Treatment | 12.70 | 44.61 | 94.18 |
| 15 - Day Treatment | 12.88 | 44.93 | 95.33 |

Table 2 shows the chicken’s body weight gain of the control group, the 7-day treatment group, and the 15-day treatment group over the span of 42 days. For all weight measurements, FPS administration leads to higher weight gains compared to control chickens. The gains in body weight are significantly higher in FPS treated chickens in comparison to untreated chickens, even though the treatment is only applied for a short duration of seven or 15 days and even though the total amount of food consumed was about the same. FCR is also seen to decrease significantly in both treatment groups compared to the non-treated control as shown in Table 3, which indicates significantly greater feed efficiency.

TABLE 2a

Body Weight Gain Comparison of Control, 7-Day and 15-Day Treatment
Weight Gain (kg)

| | | | |
|---|---|---|---|
| Age (days) | 15 | 28 | 42 |
| Control | 0.425 | 1.225 | 2.371 |
| 7- Day Treatment | 0.426 | 1.283* | 2.457* |
| 15 - Day Treatment | 0.440 | 1.331* | 2.465* |

* p < 0.05 compared to control

TABLE 2b

Body Weight Gain Percent Increase for 7-Day and 15-Day Treatment vs Control
Weight Gain (% increase vs. control)

| | | | |
|---|---|---|---|
| Age (days) | 15 | 28 | 42 |
| 7- Day Treatment | 0.23% | 4.42% | 3.49% |
| 15 - Day Treatment | 3.53% | 7.02% | 3.96% |

TABLE 3a

Calculated FCR Comparison of Control, 7-Day Treatment, and 15-Day Treatment
Calculated FCR

| | | | |
|---|---|---|---|
| Age (days) | 15 | 28 | 42 |
| Control | 1.228 | 1.542 | 1.663 |
| 7 - Day Treatment | 1.234 | 1.446* | 1.606* |
| 15 - Day Treatment | 1.207 | 1.430* | 1.617* |

* p < 0.05 compared to control

TABLE 3b

Percent Change in FCR for 7-Day Treatment, and 15-Day Treatment vs Control
FCR (% change vs. control)

| | | | |
|---|---|---|---|
| Age (days) | 15 | 28 | 42 |
| 7- Day Treatment | 0.49% | -6.23% | -3.43% |
| 15 - Day Treatment | -1.71% | -7.26% | -2.77% |

### Example 3: Challenge Trials

Challenge trials are carried out to determine whether oral administration of compositions comprising dried cell components and supernatant from a Faecalibacterium prausnitzii culture (FPS-1) can reduce pathogen load in broilers and thus reduce downstream poultry meat contamination and human infection. Simultaneously, FCR is measured to assess the potential for FPS to improve feed efficiency.

Salmonella is endemic in many farm and flock settings and can be acquired both vertically and horizontally in broilers. The challenge trial tests whether FPS can eliminate or reduce Salmonella load in broilers.

In one trial scenario, day-old, commercial, unvaccinated broiler chicks (Ross 308, Aviagen, Huntsville, AL) are allocated into floor pens in an isolation farm equipped with provisions for age-appropriate temperatures and bedding. The birds have access to ad libitum feed (Blue Seal Feeds Inc., Londonderry, NH) and water. All the experiments with birds are conducted with the approval of IACUC Institutional Animal Care and Use Committees.

In one trial scenario, FPS is supplemented through the feed for the entire 21 day trial period. On day1, birds are tested for the presence of any inherent Salmonella (n = 2 birds/group). On day 8, the birds are challenged with 1 mL of the inoculum (approximately 5.0 log₁₀ cfu of the 4-strain Salmonella Enteritidis mixture) by crop gavage. On days 1, 7, 10 and 14 post-infection (PI), birds (n=6) from each group are euthanized by carbon dioxide asphyxiation and dissected to collect organ samples for further bacteriological analysis. The experiment is replicated three times.

Cecum, small intestine, cloaca, and crop with their contents, liver, and spleen from each bird are collected in separate sterile 50 ml tubes containing 5 ml of PBS. The weighed samples are processed with a tissue homogenizer (Tissue Master, Omni International, Marietta, GA), diluted 10-fold in sterile PBS and surface plated on duplicate XLD-NA plates. When colonies are not detected after direct plating, samples are tested for surviving cells by enrichment for 48 h at 37° C. in 100 ml of selenite cysteine broth (Difco) followed by streaking on XLD-NA plates. Representative colonies from XLD-NA plates are confirmed as Salmonella with a Salmonella rapid detection kit. When colonies are not detected after direct plating, samples are tested for surviving cells by enrichment for 48 h at 37° C. in 100 ml of selenite cysteine broth followed by streaking on XLD-NA plates. Representative colonies from the plates are confirmed as Salmonella with the Salmonella rapid detection kit.

The average feed consumption and body weights of birds are determined for each experiment. Birds are weighed individually at the start and end of each experiment. The average feed consumption per bird are calculated by dividing the total amount of feed consumed per treatment group by the number of birds in the respective treatment group (Kollanoor-Johny et al., 2012a). Feed conversion ratio (FCR) is calculated for each treatment group by taking the ratio of the feed consumed by each group divided by the combined weight of all birds in that group.

Each sample is considered an experimental unit, and a completely randomized 5 X 6 X 6 X 4 factorial design are followed. Factors were five treatments (negative, positive, and FPS controls and high and low dose FPS) and six organ samples from six birds at four sampling points (days 1, 7, 10 and 14 PI). The data for bacterial counts, feed intake, and body weight from three trials for the positive control and treatment groups are averaged and analyzed with the Proc-mixed version of the Statistical Analysis Software (SAS Institute Inc., Cary, NC). Differences among the means are considered significant at P<0.05 and are detected using Fisher’s least significance difference test with appropriate corrections for multiple comparisons.

### Trial B. Campylobacter Challenge Trial

Campylobacter is known to be acquired horizontally by poultry from environmental sources (as opposed to Salmonella, which can be acquired vertically in ovo), and newborn chicks are typically not infected and believed to have passive immunity. Because of this, Campylobacter challenge trials are typically carried out by challenging week-old chicks followed by cecal extraction at a predetermined age to assess the levels of Campylobacter infection.

The Campylobacter challenge trial is conducted similarly to the Salmonella trial, using the same methods for power analysis and trial conditions with the following deviations:
Chickens are treated with FPS starting at 1 day of age and challenged with Campylobacter at 7 days of age.
Cecal extraction is carried out at 21 days of age, and levels of Campylobacter are quantified
Microaerobic culture conditions are adapted for Campylobacter.

Supplementation of the FPS formulations to broiler chicks will reduce Salmonella and/or Campylobacter colonization by antimicrobial effects, improvement of gut health and immunomodulation as well as improve feed efficiency and weight gain.

1. A composition which is a poultry food composition or composition adapted for delivery to poultry comprising cell components and supernatant from a Faecalibacterium prausnitzii culture.

2. The composition of claim 1 wherein the Faecalibacterium prausnitzii cell components and supernatant are dried.

3. The composition of claim 2, wherein the dried Faecalibacterium prausnitzii cell components and supernatant is in the form of particles having an average diameter of less than 100 microns.

4. The composition of claim 1, which is a poultry feed optimized to meet the nutritional requirements of poultry, further comprising cereal grains, oilseed meal, animal by-product meal, fats, and vitamin and mineral premixes.

5. The composition of claim 1, which is a gel spray adapted to be sprayed on chicks.

6. The composition of claim 5 which is a hydrogel spray comprising cell components and supernatant from a Faecalibacterium prausnitzii culture.

7. The composition of claim 1, which is a poultry food supplement.

8. The composition of claim 1, which is a solution, suspension or powder for admixture with drinking water.

9. The composition of claim 1, which is a composition for injection into eggs.

10. The composition of claim 1, obtained by culturing a strain of Faecalibacterium prausnitzii, centrifuging the Faecalibacterium culture to separate it into a supernatant portion and a sediment portion, drying the product, and combining it with poultry feed or a delivery vehicle selected from water, gel and feed supplement.

11. The composition of claim 1, wherein the cell components and supernatant from a Faecalibacterium prausnitzii culture are present in a concentration effective when administered to poultry to (i) enhance the feed conversion ratio of the poultry, measured with respect to weight gain, meat production, or egg production; (ii) reduce the morbidity and/or mortality in the poultry from gastrointestinal disease; (iii) reduce the pathogen contamination of meat or eggs from the poultry; (iv) improve of the meat quality of the poultry; and/or (v) to alter the poultry gut microbiota to facilitate beneficial health effects in the poultry.

12. A method for improving feed efficiency in poultry, comprising administering to the poultry an effective amount of the composition of claim 1.

13. The method of claim 12 wherein the period of administration is only during the first 30 days of life, but the improvement in feed efficiency is sustained throughout the bird’s life.

14. The method of claim 12 wherein the improved feed efficiency is measured by feed conversion ratio (FCR) with respect to weight gain and/or meat production or with respect to egg production, wherein the poultry exhibit an improvement in feed conversion ratio of at least 0.5%, relative to control poultry, which do not receive the composition.

15. The method of claim 12 wherein the administration of effective amount of the composition is by feeding the poultry a composition comprising optionally dried cell components and supernatant from a Faecalibacterium prausnitzii culture, or by spraying chicks with a gel comprising a composition comprising optionally dried cell components and supernatant from a Faecalibacterium prausnitzii culture, or by injecting eggs with a composition comprising optionally dried cell components and supernatant from a Faecalibacterium prausnitzii culture.

16. A method for reducing morbidity and/or mortality in poultry from gastrointestinal disease, comprising administering to the poultry an effective amount of the composition of claim 1.

17. A method for reducing pathogen load in poultry and/or reducing pathogen contamination of meat or eggs from poultry, comprising administering to the poultry an effective amount of the composition of claim 1, wherein the pathogen is a human bacterial pathogen selected from Campylobacter spp., pathogenic E. coli strains, Enterobacteriaceae spp. and Salmonella spp.

18. A method for improvement of meat quality in poultry, comprising administering to the poultry an effective amount of the composition of claim 1.

19. A method for alteration of poultry gut microbiota to facilitate beneficial health effects, in comparison to untreated poultry, comprising administering to the poultry an effective amount of the composition of claim 1.

20. A method of making a composition of claim 1, comprising culturing a strain of Faecalibacterium prausnitzii, centrifuging the Faecalibacterium culture, to separate it into a supernatant portion and a sediment portion, drying the product, and combining it with poultry feed or a delivery vehicle selected from water, gel and feed supplement suitable for consumption by poultry.
